(19) United States
(12) Patent Application Publication
(10) Pub. No.: US 2026/0248556 A1
(43) Pub. Date: Aug. 27, 2026

(54) TREATMENT OF ASTHMA AND COPD WITH PULSED ELECTRIC FIELD ABLATION AND PRECISION DRUG DELIVERY

(71) Applicant: Boston Scientific Medical Device Limited, Galway (IE)

(72) Inventors: Wentian Zhang, Shanghai (CN); Yao Chen, Shanghai (CN); Xing Xia, Nanjing (CN); Yinghua Wang, Shanghai (CN); Mingfeng Xie, Shanghai (CN); Cheng Zhang, Shanghai (CN)

(73) Assignee: Boston Scientific Medical Device Limited, Galway (IE)

(21) Appl. No.: 19/547,835

(22) Filed: Feb. 24, 2026

(30) Foreign Application Priority Data
Feb. 24, 2025 (CN) 202510205621.1

(51) Int. Cl. A61B 18/14; A61B 18/00

(52) U.S. Cl. CPC A61B 18/1492; A61B 2018/00154; A61B 2018/0022; A61B 2018/00541; A61B 2018/00577; A61B 2018/1435; A61B 2018/1467; A61B 2218/007

(57) ABSTRACT

Methods, systems and devices relating to pulsed-field-ablation therapy for respiratory maladies and malformations are disclosed. In some examples, an ablation catheter and methods of use are disclosed with may include an ablation catheter including a proximal shaft including an expandable scaffolding, a control knob, a distal shaft including a distal tip disposed proximate the distal shaft distal end. Systems, methods and devices may further incorporate a control knob disposed proximate the proximal shaft and the distal shaft. The ablation catheters described herein may include one or more electrodes, and both the proximal shaft and the distal shaft may include at least one lumen for the passage of one or more electrodes therethrough.

### CROSS-REFERENCE TO RELATED APPLICATIONS

This application claim priority to Chinese Application Number 202510205621.1, filed on February 24, 2025, the entire disclosures of which are hereby incorporated by reference.

### TECHNICAL FIELD

The present disclosure pertains to ablative treatment systems, methods and devices which may be incorporated and/or included with drug and/or therapeutic delivery for the treatment of respiratory maladies and malformations.

### BACKGROUND

Asthma and chronic obstructive pulmonary disease (COPD) both result in a reduction of ciliated cells, which are responsible for removing harmful substances. These cells are replaced by goblet cells, which produce mucus and are the primary source of sputum secretion. The increase in the number of goblet cells, coupled with their enhanced secretory function and reduction in ciliated cells, weakens the mucus clearance function, thereby leading to airway narrowing.

Patients with asthma and COPD experience symptoms such as difficulty breathing, excessive sputum production, and coughing, which significantly impact their quality of life. Moreover, asthma and COPD increase the likelihood of lung infections, thereby elevating the risk of acute and severe hospitalization. Asthma and COPD may co-exist or overlap, particularly in smokers and the elderly.

### TECHNICAL FIELD

The present disclosure pertains to ablative treatment systems, methods and devices which may be incorporated and/or included with drug and/or therapeutic delivery for the treatment of respiratory maladies and malformations.

### BACKGROUND

Asthma and chronic obstructive pulmonary disease (COPD) both result in a reduction of ciliated cells, which are responsible for removing harmful substances. These cells are replaced by goblet cells, which produce mucus and are the primary source of sputum secretion. The increase in the number of goblet cells, coupled with their enhanced secretory function and reduction in ciliated cells, weakens the mucus clearance function, thereby leading to airway narrowing.

Patients with asthma and COPD experience symptoms such as difficulty breathing, excessive sputum production, and coughing, which significantly impact their quality of life. Moreover, asthma and COPD increase the likelihood of lung infections, thereby elevating the risk of acute and severe hospitalization. Asthma and COPD may co-exist or overlap, particularly in smokers and the elderly.

Asthma is a common chronic respiratory disease characterized by the proliferation of submucosal glands and an increase in mucus-secreting goblet cells, accompanied by a reduction in ciliated cells. The mucus contains a higher concentration of acidic proteins, fibrin, extravasated albumin, and other tissue injury proteins, which transforms the mucus into a gel-like substance that forms mucus plugs, obstructing the airways. Asthma affects approximately 262 million people worldwide with an estimated 1000 deaths each day.

The most common treatment for asthma is pharmacotherapy, which can be categorized into reliever medications and controller medications. Reliever medications can dilate spasmodic airways, providing rapid symptom relief. Given the unpredictability of asthma attacks, patients are advised to always carry these medications. Controller medications are primarily used for the daily management and treatment of asthma to reduce symptoms and the frequency of attacks. These medications need to be used regularly and consistently over the long term. Discontinuation without medical advice can lead to more frequent recurrences or severe asthma attacks. However, prolonged or excessive use of these medications may result in side effects such as hypertension, diabetes, arrhythmias, and osteoporosis.

In addition, for asthma patients who have been on the GINA (Global Initiative for Asthma) step 4 or step 5 treatment regimen for six months or longer without achieving adequate control, or those who lose control during step-down therapy (i.e., during oral steroid tapering) despite being on the GINA step 4 or step 5 regimen, bronchial thermoplasty (BT) may be considered. BT is a non-pharmacological intervention performed under bronchoscopy. It reduces the amount of airway smooth muscle, decreases bronchial contractility, lowers airway hyperresponsiveness, improves asthma control, enhances patients’ quality of life, and reduces the need for medication. The short-term efficacy and safety of BT are increasingly supported by research, but its long-term efficacy requires further investigation. Additionally, attention must be paid to potential complications, such as bleeding, bronchial perforation, and vocal cord damage.

Chronic bronchitis (CB) and pulmonary emphysema are among the most significant structural changes in chronic obstructive pulmonary disease (COPD). CB is often associated with prolonged exposure to smoke from cigarettes, biomass fuels, and other irritants. When the airway mucosa is irritated, the number of ciliated cells responsible for clearing harmful substances decreases and is replaced by mucus-producing goblet cells, which are the primary source of sputum secretion. The increase in goblet cell numbers and their enhanced secretory function, coupled with the reduction in ciliated cells, weakens the mucus clearance function. The progression of CB leads to airway obstruction and gas trapping. The terminal bronchiolar structures and alveoli, like balloons, become increasingly inflated. Once the tension of the alveolar walls is exceeded, potential irreversible structural changes occur, resulting in the formation of emphysema characterized by overinflated alveoli. Currently, there are more than 480 million people afflicted with COPD worldwide.

The treatment of COPD primarily focuses on symptom control through medication, though the efficacy is often suboptimal and adverse drug reactions must be monitored. Surgical or interventional treatments, such as surgical lung volume reduction and steam ablation, can provide significant clinical benefits in managing chronic obstructive pulmonary disease. However, these procedures may also be associated with serious complications, including airway stenosis, pulmonary embolism, pulmonary hemorrhage, pneumothorax, and respiratory failure.

Pulsed field ablation (PFA) is a novel non-thermal ablation therapy that has emerged in recent years. It works by generating and delivering short-duration, intermittent, high-energy pulses, which cause changes in the ion channels of cell membranes and disrupt the phospholipid bilayer in the tissues exposed to an effective electric field. This increases the permeability of the cell membrane to molecules, ultimately leading to cell death. Due to its non-thermal and expected tissue-selective ablation characteristics, PFA can ablate over-proliferated goblet cells, reduce mucus secretion in the airways, and alleviate obstruction without causing damage to the surrounding tissues. Moreover, since the depth of mucosal ablation by pulsed field energy is controllable and limited to the superficial mucosa and submucosa, it does not affect the deeper smooth muscle and cartilage, thereby preserving the mucosal regeneration and repair capabilities. Therefore, for patients with high airway mucus secretion and significant proliferation of mucus-producing cells, this therapy can reduce the source of mucus, thereby decreasing excessive mucus secretion in the airways, leading to clinical benefits.

PFA has unique advantages over thermal ablation regarding at least its cell targeting feature. Goblet cells are more sensitive to PFA as goblet cells are larger than normal columnar ciliated cells, since it is easier for cells with a larger size to reach the cell membrane electroporation threshold and exhibit stronger electrical sensitivity under the same electric field strength.

Currently, the treatment of asthma and COPD primarily focuses on symptom control through medication. However, inhalation limits the absorption and only about 10% of the aerosol enters the respiratory tract while the remainder is swallowed and may be absorbed in the intestinal tract with consequent systemic side effects, including muscular tremor, tachycardia, hypokalemia and arrhythmias.

By delivering PFA energy to one or more target lesions, the number of goblet cells may be decreased, which reduces the source to produce mucus and thereby mitigates the airway obstruction, a symptom of asthma and COPD. Moreover, since airway obstruction is usually caused by chronic inflammation, excessive secretion of mucus may result in airway wall remodeling. The use of anti-inflammatory drugs is also an effective way to relieve airway obstruction symptoms. Direct airway wall infusion of drugs may provide elevated drug concentration at the target while minimizing systemic side effects. Moreover, the rate of drug absorption may be improved due to small pores generated on the cell membrane following PFA.

Thus, there exists an unmet need for more effective techniques, methods and systems to treat asthma, COPD and related diseases, malformations and/or maladies. More particularly through innovative uses of PFA and related modalities, techniques, devices and methods.

### SUMMARY

The present disclosure provides methods, devices and systems that substantially advance the efficacy and safety of treatment for respiratory maladies and/or malformations, including but not limited to chronic bronchitis (CB), chronic obstructive pulmonary disease (COPD), asthma, and the like.

In at least one example, the disclosure provides an ablation catheter, including: a proximal shaft including an expandable scaffolding, a control knob, a distal shaft including a distal tip disposed proximate the distal shaft distal end. In this and other examples, the control knob may be disposed proximate the proximal shaft and the distal shaft. The ablation catheter of this and other examples may further include one or more electrodes, and both the proximal shaft and the distal shaft may include at least one lumen for the passage of one or more electrodes therethrough.

Alternatively, or additionally, the proximal shaft and the distal shaft may each include three or more lumens for the passage of three or more electrodes.

Alternatively, or additionally, the proximal shaft and the distal shaft may each include one or more lumens for the passage of one or more drugs and/or one or more therapeutics.

Alternatively, or additionally, the distal tip may include one or more electrodes disposed proximate the distal tip and/or disposed upon the distal tip.

Alternatively, or additionally, the expandable scaffolding may include an expandable balloon.

Alternatively, or additionally, the expandable scaffolding may include one or more electrodes disposed within and/or upon the expandable scaffolding.

Alternatively, or additionally, the expandable scaffolding may include one or more drug coatings disposed thereon and/or one or more drug dispensing elements disposed thereon or within.

Alternatively, or additionally, the ablation catheter may be configured and/or otherwise adapted to be operably connected to an electrical pulse generator, such that the ablation catheter is configured and/or otherwise adapted to deliver pulsed electrical energy through the one or more electrodes.

Alternatively, or additionally, the one or more electrodes may be disposed in an array of electrodes, whereby the array of electrodes may be one or more of a spiral array, a linear array, a curvilinear array, and an interrupted array.

In other non-limiting examples, the disclosure provides methods of treating respiratory maladies and malformations. The methods of this and other examples may include: deploying a first catheter into a lung of a patient or subject; suctioning sputum from a lung of a patient or subject by a suctioning means; activating one or more electrodes disposed on or within the first catheter, thereby delivering pulsed electrical therapy for ablation of a treatment site within a lung of a patient or subject; and delivering a drug and/or therapeutic to the treatment site.

Alternatively, or additionally, the methods of this and other examples may include delivering one or more drugs and/or therapeutics via one or more lumens within the first catheter.

Alternatively, or additionally, the methods of this and other examples may include delivering one or more drugs and/or therapeutics to the treatment site via the first catheter; whereby the one or more drugs and/or therapeutics are selected from one or more of: one or more drug and/or therapeutic coatings, one or more drug and/or therapeutic dispensing elements, one or more lumen-delivered drugs and/or therapeutics, one or more electrically delivered drugs and/or therapeutics, and one or more pulse-delivered drugs and/or therapeutics.

Alternatively, or additionally, the methods of this and other examples may include delivering anti-inflammatory drugs and/or therapeutics after or concurrent with ablation.

Alternatively, or additionally, the methods of this and other examples may include whereby the one or more electrodes include two or more electrodes disposed upon and/or within an expandable scaffolding operably coupled to both the first catheter and a control knob operably coupled to the first catheter, the methods of this and other examples may include spreading apart the two or more electrodes via an expandable scaffolding operably coupled to the first catheter and in response to action of a control knob operably coupled to the first catheter.

In yet other non-limiting examples, the disclosure provides a pulsed-electric-field ablation catheter, including: a proximal shaft, a control knob, a distal shaft including a distal tip disposed proximate the distal shaft distal end, whereby the control knob is disposed proximate the proximal shaft and the distal shaft; whereby the ablation catheter includes one or more electrodes disposed upon or within an expandable framework, whereby the expandable framework further includes one or more drug coatings or one or more drug dispensing elements disposed upon or within the expandable framework, whereby both the proximal shaft and the distal shaft include at least one common lumen for the passage of one or more electrodes, one or more drugs, and one or more therapeutics therethrough.

### BRIEF DESCRIPTION OF THE DRAWINGS

The disclosure may be more completely understood in consideration of the following detailed description in connection with the accompanying drawings, in which:

FIG. 1 shows an example of lung anatomy of a prospective subject and/or patient, comparing un-enflamed lung tissue with enflamed lung tissue.

FIGS. 2A and 2B illustrate examples of an ablation catheter in accordance with at least one embodiment.

FIG. 3 illustrates the interconnectivity of features of the disclosure described herein.

FIGS. 4A and 4B illustrate additional and/or alternative examples of an ablation catheter in accordance with at least one embodiment.

FIGS. 5A and 5B illustrate a control knob in accordance with at least one embodiment.

FIGS. 6A and 6B illustrate ablation catheters in accordance with at least one embodiment.

FIG. 7 illustrates a portion of an ablation catheter in accordance with at least one embodiment.

FIGS. 8A, 8B, and 8C illustrate additional and/or alternative ablation catheters in accordance with at least one embodiment.

FIGS. 9A and 9B illustrate constructive features of ablation catheters disclosed herein and in accordance with at least one embodiment.

FIGS. 10A and 10B illustrate constructive features of ablation catheters disclosed herein and in accordance with at least one embodiment.

FIG. 11 illustrates an example flow chart in accordance with at least one embodiment and/or methods disclosed herein.

### DETAILED DESCRIPTION

For the following defined terms, these definitions shall be applied, unless a different definition is given in the claims or elsewhere in this specification.

All numeric values are herein assumed to be modified by the term “about,” whether explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more features, structures, and/or characteristics. However, such recitations do not necessarily mean that all embodiments include the features, structures, and/or characteristics. Additionally, when features, structures, and/or characteristics are described in connection with one embodiment, such features, structures, and/or characteristics may also be used connection with other embodiments whether explicitly described unless clearly stated to the contrary.

It can be appreciated that the term/phrase “ablation catheter” may alternately refer to the terms/phrases: “pulsed-field-ablation catheter”, “pulsed field ablation catheter”, “pulsed-electric-field ablation catheter”, “pulsed electric field ablation catheter”, “pulsed-field catheter”, “pulsed field catheter”, “pulsed-electric-field-ablation catheter”, “pulsed electric field ablation catheter”, “pulsed electrical therapy ablation catheter”, “pulsed electrical therapy catheter”, and/or any term/phrase of the like and/or any suitable and/or feasible term/phrase known in the art.

The following detailed description should be read with reference to the drawings in which similar structures in different drawings are numbered the same. The drawings, which are not necessarily to scale, depict illustrative embodiments and are not intended to limit the scope of the disclosure.

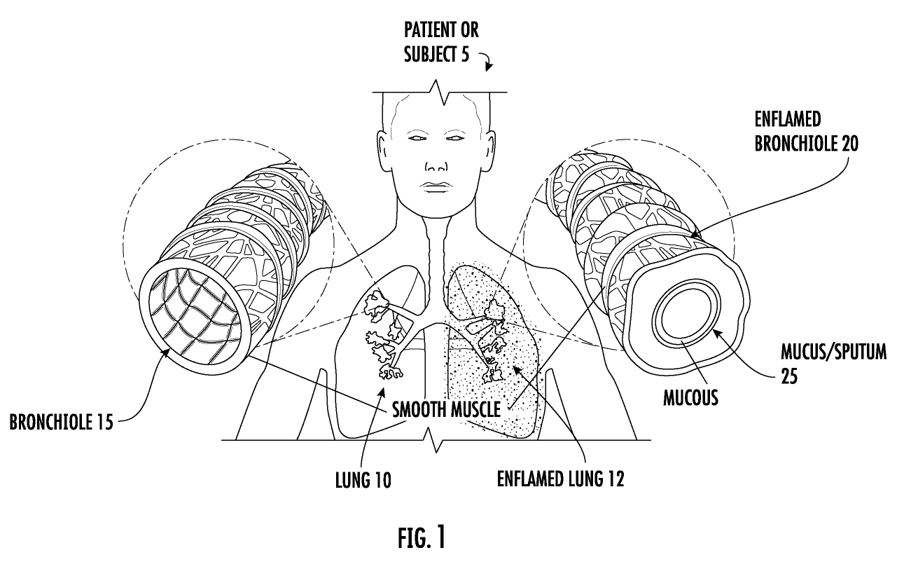

FIG. 1 illustrates example respiratory anatomy of a patient or subject 5 in which the left side of the FIGURE(anatomical right) depicts a lung 10 devoid of advanced inflammation. As shown in the left-hand close-up view of FIG. 1, a bronchiole 15 with minimal or no inflammation and therefore an unrestricted (i.e., not narrowed) internal diameter is present. This is contrasted with the right side of FIG. 1 (anatomical left), which depicts an enflamed lung 12 with an enflamed bronchiole 20 presenting corresponding mucus buildup 25.

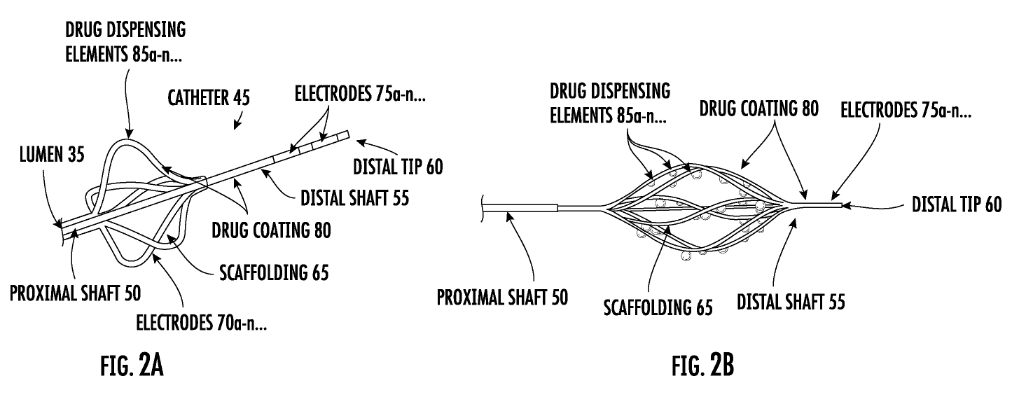

Shown in FIGS. 2A and 2B are examples of ablation catheters of the present disclosure which, at least in a non-limiting sense, are intended to treat respiratory maladies and malformations including those shown in FIG. 1. Turning to FIG. 2A, an ablation catheter 45 is shown with a proximal shaft 50 and a distal shaft 55 coupled distally thereto. In this and other examples, ablation catheter 45 may include a lumen 35 and may include more than one lumen for the passage of electrodes, electrical contacts, electrical elements, electrical wires, ablation elements, ablation wires, drugs, therapeutics, and/or agents as will be described further herein.

The proximal shaft 50 and/or the distal shaft 55 of the ablation catheter 45 may be coated with one or more of a drug and/or therapeutic and/or agent, and/or may be coated with one or more drugs and/or therapeutics and/or agents provided in a coating 80. In this and other examples, the ablation catheter 45 may be coated with a corticosteroid, a bronchodilator (including but not limited to β2-agonists, anticholinergic antimuscarinic agents, methylxanthines and/or the like), glucocorticoids, and/or other types of medication, such as but not limited to: vaccines, antibiotics, α1-antitrypsin augmentation therapy, mucolytic agents, antioxidants, immunoregulators, antitussives and/or vasodilators. In this and other examples, the drugs and/or therapeutics and/or agents (or the like) may be delivered through a delivery channel integrated in the ablation catheter 45 as will be described herein.

Also shown in at least FIG. 2A is scaffolding 65. In this and other examples, scaffolding 65 may be expandable, compressible, collapsible, inflatable, contractile, and/or the like. Scaffolding 65 may further include electrodes 75. In non-limiting examples, scaffolding 65 may include one or more electrodes 70, which may be denoted as electrode 70a, electrode 70b, electrode 70c, etc. Electrodes 70 may be embedded within the scaffolding 65, disposed within the scaffolding 65, disposed upon scaffolding 65, disposed proximate to scaffolding 65, disposed distal to scaffolding 65, disposed proximal to scaffolding 65, and/or any combination of the aforementioned. It can be appreciated that scaffolding 65 (in addition to other scaffolding examples disclosed herein) may be coated with a corticosteroid, a bronchodilator (including but not limited to β2-agonists, anticholinergic antimuscarinic agents, methylxanthines and/or the like), glucocorticoids, and/or other types of medication, such as but not limited to: vaccines, antibiotics, α1-antitrypsin augmentation therapy, mucolytic agents, antioxidants, immunoregulators, antitussives and/or vasodilators and/or the like.

Ablation catheter 45 may further include electrodes 75 on and/or along the distal shaft 55 and may further or alternatively include electrodes 75 on a distal tip 60 of the ablation catheter 45 which may be molded onto, adhered, snap-fitted, interference-fitted, bonded to, made integral with, and/or otherwise coupled to distal shaft 55. Electrodes 75 may be provided as multiple electrodes, such as electrodes 75a, 75b, 75c, etc. It is further and/or alternatively contemplated that scaffolding 65 may include drug dispensing elements 85, and may include one or more drug dispensing elements (85a, 85b, 85c, etc.). Drug dispensing elements 85 will further be described herein and may include (but are not limited to) drug elution elements, selectively absorbable drug dispensing elements, pressure-aided drug dispensing elements, time-release drug dispensing elements, or the like, or any combination or permutation of the aforementioned.

FIG. 2B illustrates an alternative and/or additional embodiment of an ablation catheter 45 in which the scaffolding 65 is shown in an expanded and/or inflated state. As shown in FIG. 2B, scaffolding 65 may include one or more drug dispensing elements (85a, 85b, 85c, etc.) and/or one or more coatings 80. Coatings 80 may include, but are not limited to drug coatings, therapeutic coatings, agent coatings, therapeutic agent coatings, anti-inflammatory coatings, vasodilating coatings, or any combination or permutation of the, or any of the like.

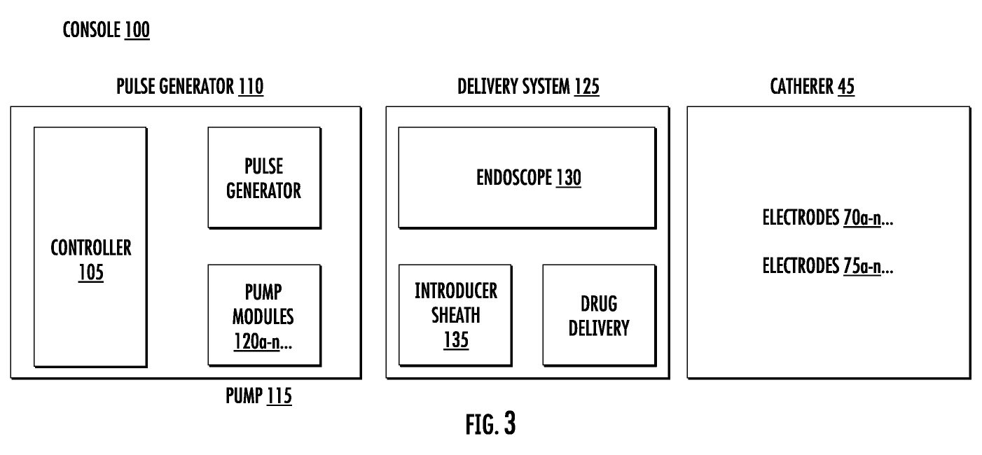

FIG. 3 shows a chart displaying the interconnectivity of several features of the present disclosure. As shown in FIG. 3, and by non-limiting example, a console 100 may be operably connected to and/or otherwise coupled with any of the devices and/or systems of the present disclosure. Furthermore, a controller 105 may be in operable connection with any of the devices and/or systems of the present disclosure and may report and/or display its status on console 100. Further shown in FIG. 3 is a pulse generator 110 which may be communicable and/or operably connected to any of the devices and/or systems of the present disclosure, including all electrodes disclosed herein. Console 100 may further display and/or report the status of pump modules 120, of which there may be one or more pump modules denoted as 120a, 120b, 120c, etc. Pump modules may also communicate with a pump 115, which may be operably connected and/or otherwise communicable with any of the devices and/or systems of the present disclosure, which may include operable communication and/or fluid communication with the ablation catheters disclosed herein.

Also shown in FIG. 3 are non-limiting examples of devices which may be operably connected to, coupled with, and/or otherwise communicable with any of the devices and/or systems of the present disclosure, including but not limited to an endoscope 130 and introducer sheath 135. These features, as well as any of the additional and/or alternative features disclosed herein may form a delivery system 125. As shown in FIG. 3, each of the devices, at least in a non-limiting sense, may further communicate and/or couple with the ablation catheters and/or ablation elements and/or electrical elements and/or drug dispensing elements of the present disclosure.

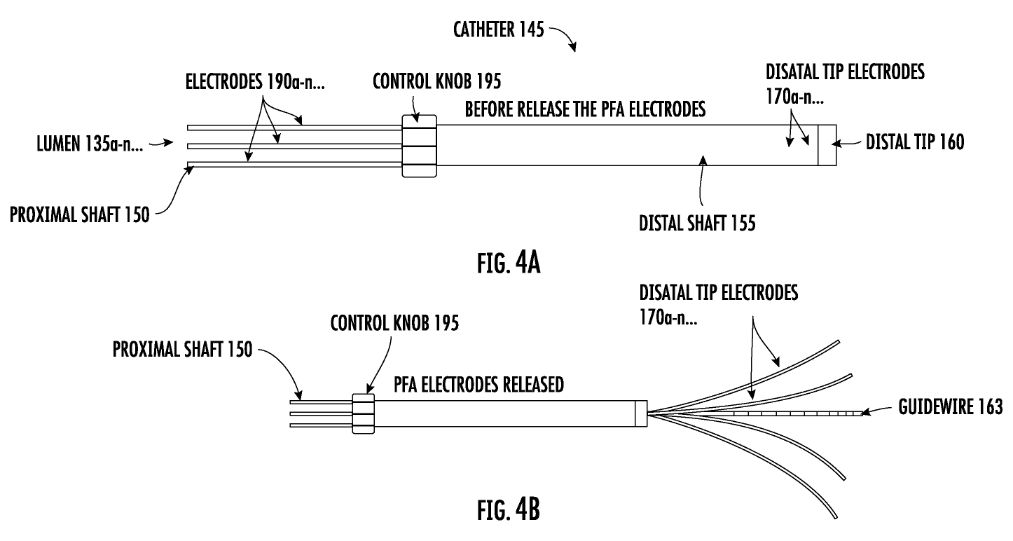

FIG. 4A depicts another non-limiting example of the disclosure herein. As shown, one or more lumens 135a, 135b, 135c, etc. may be provided and may run and/or pass through at least proximal shaft 150, control knob 195, distal shaft 155 and distal tip 160. In this and other examples, the one or more lumens 135 may create one or more common lumens. In other words, the one or more lumens 135 may be fluidly communicable throughout the catheter 145, thus forming one or more common lumens. In examples with a single lumen, the single lumen may form a common lumen extending through two or more of the proximal shafts 150, control knob 195, distal shaft 155 and distal tip 160.

It can be seen in at least FIG. 4A that a series of electrodes 190a, 190b, 190c etc. may pass along and/or through one or more lumens 135. It is also contemplated that one or more lumens 135 may allow passage and/or substantially contain one or more electrodes 190. It is also contemplated that each of the one or more lumens 135 may allow passage and/or substantially contain one electrode 190 per lumen 135. Electrodes 190 may emerge from the distal tip 160 of ablation catheter 145 as shown in FIG. 4B as distal tip electrodes 170. Distal tip electrodes 170 may also be integrated with electrodes 190 and/or provided as a multi-piece construction with electrodes 190.

Control knob 195, as will be described further herein may be disposed proximate to both the proximal shaft 150 and the distal shaft 155. In other words, control knob 195 may be located in between the proximal shaft and the distal shaft or may be located at any point between the proximal most edge of the proximal shaft 150 and/or the distalmost edge of the distal shaft 155 which may include distal tip 160. Further shown in FIG. 4B, but not intending to be limiting, is guidewire 163. Guidewire 163 may serve to guide catheter through anatomical structures within a patient and/or subject. Guidewire 163 may be in communication with a controller, electrical source, and/or electrical generator and/or electrical pulse generator and/or pulse generator as described herein. Further, and additionally non-limiting, guidewire 163 may include one or more electrodes and/or one or more electrical contacts and/or one or more electrical elements, such that guidewire 163 may provide therapy and/or ablation to a patient and/or subject in addition or alternative to the known functions of a guidewire.

The treatment area of a patient or subject may be identified through clinical imaging, and the ablation catheter 145 may be deployed to the appropriate position via bronchoscopy or other like procedure. Subsequently, under the guidance of imaging techniques (such as ultrasound and/or CT), the pulsed-field-ablation catheter electrodes 170a-n may be released into different tracheal branches to perform ablation therapy simultaneously upon regions of desired treatment (i.e., treatment sites). The number of electrodes 170a-n may be determined based upon the condition of the treatment site. In other words, factors such as size and/or number of lesion(s) may determine the surface area and/or volume of the treatment site in which a physician and/or practitioner would target and focus ablation via the pulsed-field-ablation catheter 145. The ablation parameters for pulsed field ablation may be determined based upon the lethal threshold of goblet cells and the reversible electroporation threshold of airway smooth muscle cells at and/or around the treatment site. The drugs, therapeutics, and/or agents released at this point (e.g., treatment site) may be effectively absorbed by the cells, thereby enhancing drug absorption and simultaneously controlling the side effects by reducing the dosage of the drugs, therapeutics, and/or agents.

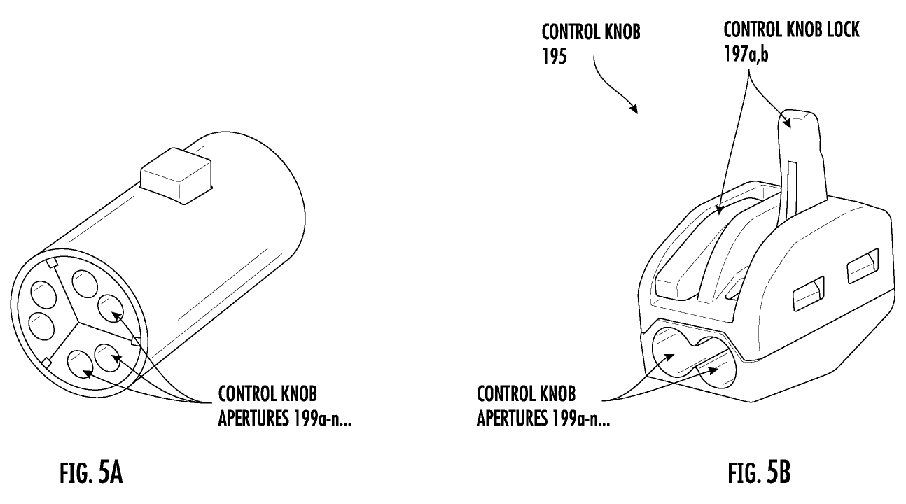

FIG. 5A illustrates an example of a control knob 195 in accordance with embodiments of the present disclosure. As seen in FIG. 5A, control knob 195 may include one or more control knob apertures 199 (199a, 199b, 199c, etc.) which may allow for the passage therethrough of electrodes, electrical elements, therapeutic elements, drugs, therapeutics, therapeutic agents, agents, anti-inflammatory products, and/or any of the like, any of the aforementioned, and/or any combination or permutation of the aforementioned.

Further shown in FIG. 5B is another example of a control knob 195 which employs one or more control knob locks 197a, 197b. One or more control knob locks 197a, 197b may be utilized for positioning of the ablation catheters disclosed herein, for positioning one or more guidewires, one or more electrodes, one or more shafts, and/or one or more elements and/or features of the devices disclosed herein feasible for communication with any of the control knobs disclosed herein.

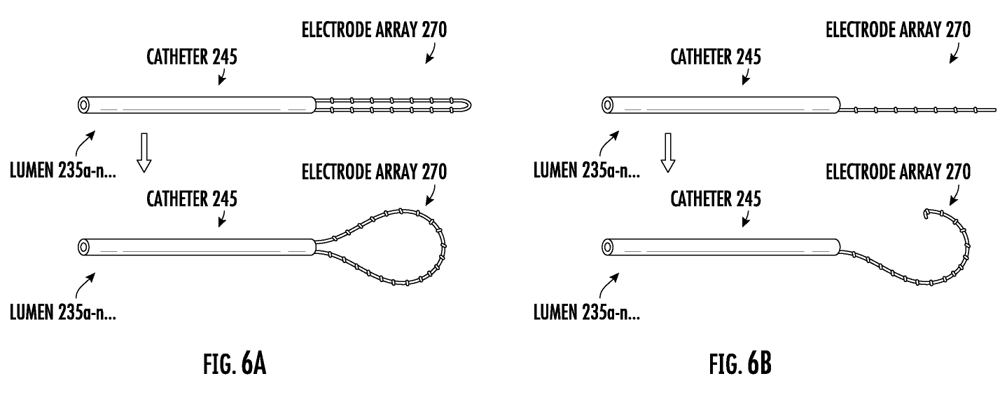

FIG. 6A illustrates another non-limiting example of the devices disclosed herein. As shown, an ablation catheter 245 includes an electrode array 270 disposed therein, and/or upon, and or in communication with one or more lumens 235. As shown in the progression through FIGS. 6A and 6B, electrode array 270 may be provided in multiple configurations. Not limited by the depictions in FIGS. 6A and 6B, electrode array 270 may be provided in a spiral array, a helical array, a linear array (see top of FIG. 6B), a curved and/or curvilinear array (see bottom of FIG. 6B) and/or may be provided in myriad geometrical arrays, including but not limited to the elliptical array shown at the bottom of FIG. 6A. In yet other non-limiting examples, electrode array 270 and/or any other electrode array of the disclosed examples may be provided in an interrupted array. In other words, electrode array 270 and/or any other example electrode array may be discontinuous, and/or provided in parts separated over and/or by any element, surface, length, or dimension of the ablation catheters disclosed herein. Optionally, a pull wire and/or steering mechanism (not shown) or other structure may be attached to electrode array 270 to allow electrode array 270 to bend, flex, expand, contract, collapse and/or conform to any geometrical configuration.

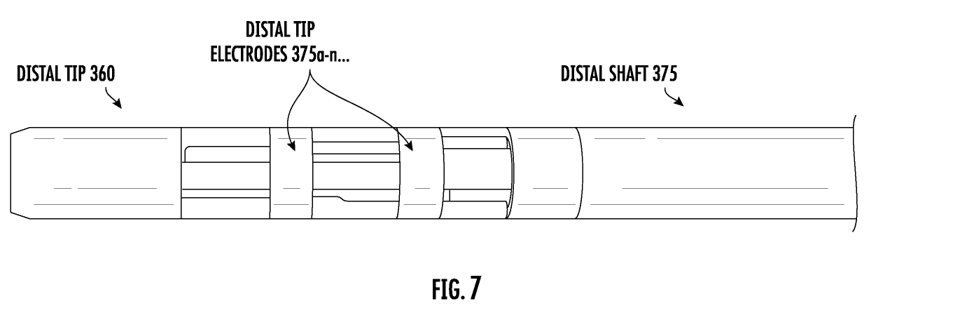

FIG. 7 illustrates a non-limiting example of a distal end configuration of the ablation catheters described herein. As shown in FIG. 7, distal shaft 375 may include a series of electrodes, such as distal tip electrodes 375 which may be located proximate to one or both distal shaft 375 and distal tip 360. In other non-limiting examples, distal tip electrodes 375 (as well as other electrodes contemplated herein) may be provided in a series, an array, and/or other known configurations of electrodes known in the art. In yet other non-limiting examples, distal tip electrodes (as well as other electrodes contemplated herein) may be provided as one or more ring electrodes, one or more partial-ring electrodes, one or more annular electrodes, one or more partially annular electrodes, one or more protruding electrodes, one or more recessed electrodes, and/or any combination or permutation of the aforementioned.

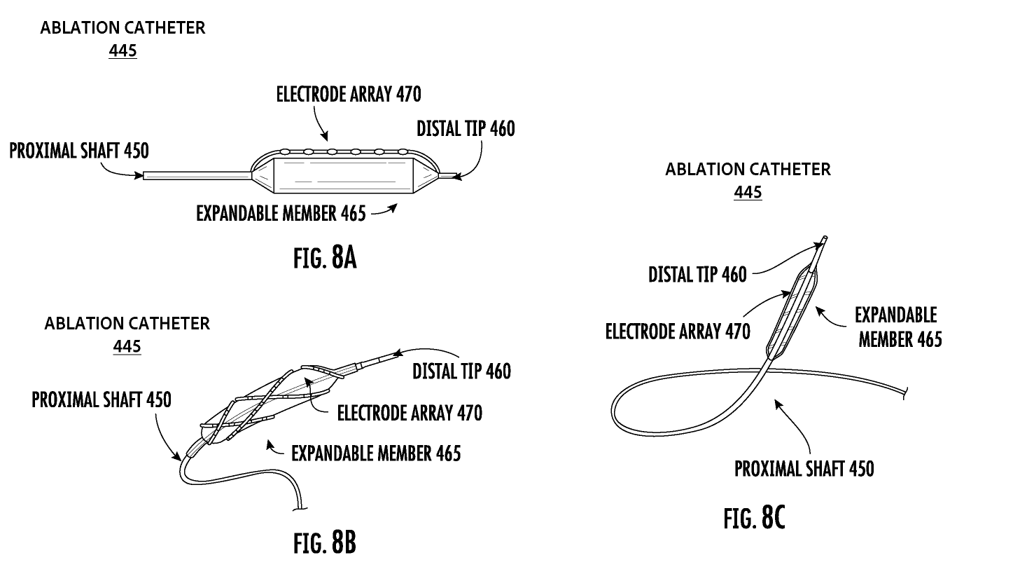

FIG. 8A shows another non-limiting example of an ablation catheter disclosed herein. In FIG. 8A, an electrode array 470 is shown disposed within an expandable member 465. Electrode array 470 (and other electrodes and/or electrode arrays described herein) may be disposed within and/or upon expandable member 465 in myriad arrays and arrangements, including but not limited to a spiral array, a helical array, a linear array, a curvilinear array, an interrupted array, and/or any combination or permutation of the aforementioned. Expandable member 465 may take myriad forms, including but not limited to an expandable balloon, an expandable member, an expansible member, an expandable scaffolding, an expandable bladder, an inflatable member, a series and/or collection of expandable struts and/or arms, an expandable material, a temperature-dependent expandable material, or the like and/or any combination or permutation of the aforementioned.

As shown in FIG. 8B, electrode array 470 is provided about expandable member 465 in a spiral array, or in the alternative, a helical array as the electrode array 470 wraps around the diameter and/or circumference of the expandable member 465. Further shown in FIG. 8C is an ablation catheter 445 wherein the expandable member 465 is in a collapsed configuration, or in a semi-collapsed configuration. As seen by FIG. 8C, electrode array 470 may be a series of spaced-apart electrodes. However, it is also contemplated that electrode array 470 may be discontinuous, interrupted, and/or provided in a spiral array, a helical array, or any combination or permutation of the arrangements.

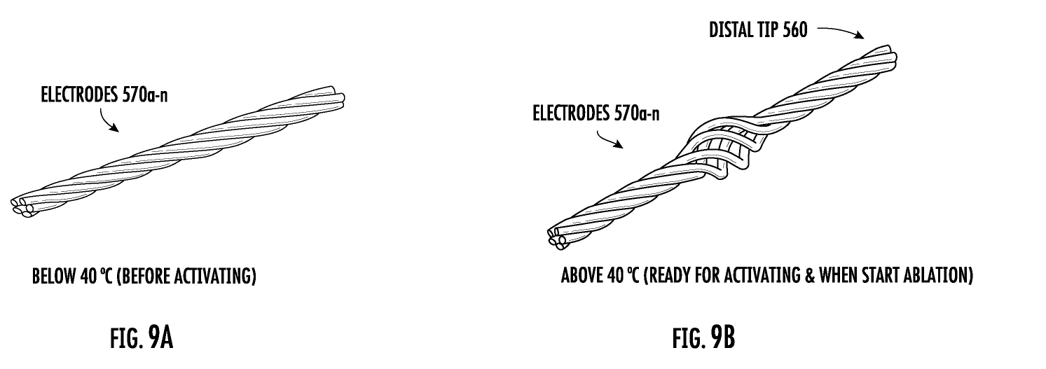

FIGS. 9A and 9B illustrate another non-limiting example of a distal tip and electrodes that may be incorporated into any example described herein. In FIG. 9A, electrodes 570 may be provided as a temperature-dependent wire (also known as an ablation wire), or series and/or interweaving of temperature-dependent wires and/or ablation wires which may experience no change in state, size, shape and/or activation in temperatures below 40 degrees Celsius (by non-limiting example). Progressing to FIG. 9B, when temperatures in the surrounding environment (i.e., temperature provided by the controller, pulse generator, electrodes, electrical elements, and/or interior anatomy of a patient or subject) exceed 40 degrees Celsius, the ablation wire including electrodes 570 may activate and/or conform and/or change shape and/or change size to provide therapy, provide ablation, provide heating, provide electrical therapy, and/or perform any of the functions ascribed to the electrodes disclosed herein. It can also be seen from FIG. 9B that ablation wire, including electrodes 570, may preferentially deform and/or expand and/or change shape to provide greater efficacy in treatment and/or better targeted treatment. Ablation wires, including electrodes 570, may be made of any feasible material known in the art, including but not limited to nitinol, titanium, nitinol-titanium alloys, and/or other like alloys.

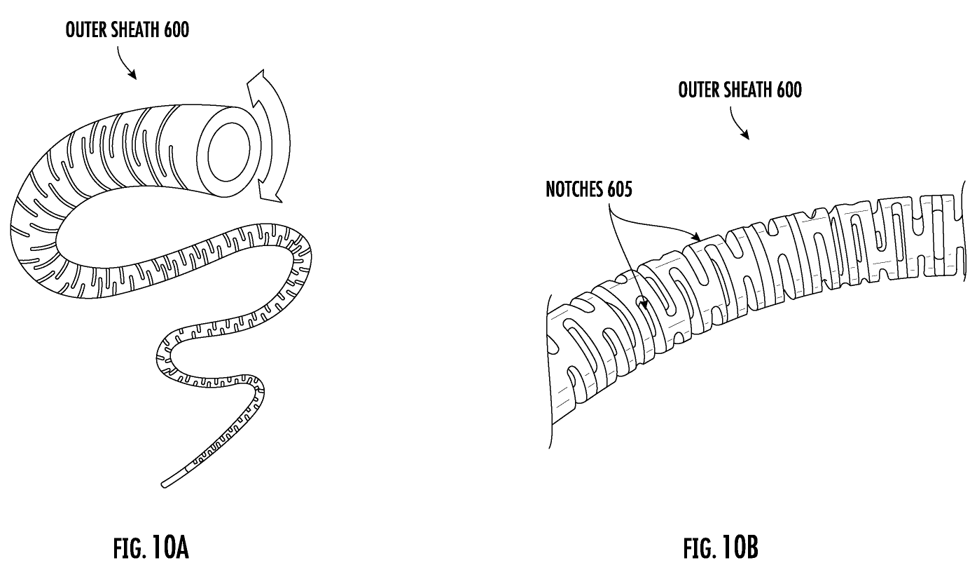

FIGS. 10A and 10B illustrate an example of an outer sheath 600 which may be incorporated with any example or embodiment disclosed herein. Outer sheath 600 may serve myriad purposes, including but not limited to providing insulation over, upon, and/or for the electrodes disclosed herein. In other non-limiting examples, outer sheath 600 may provide selective insulation over, upon, and/or for the electrodes disclosed herein such that therapy can be better targeted at and/or upon a treatment site, such as a treatment site within a patient or subject. Outer sheath 600 may further include notches 605, and/or may alternatively include knurls, depressions, grooves, concavities, convexities, discontinuities, valleys, peaks, striations, or other surface and/or depth features that may allow improved navigation of the devices disclosed herein. Outer sheath 600 may be formed of any one or more materials known in the art, including but not limited to insulative materials and/or semi-conductive materials known in the art.

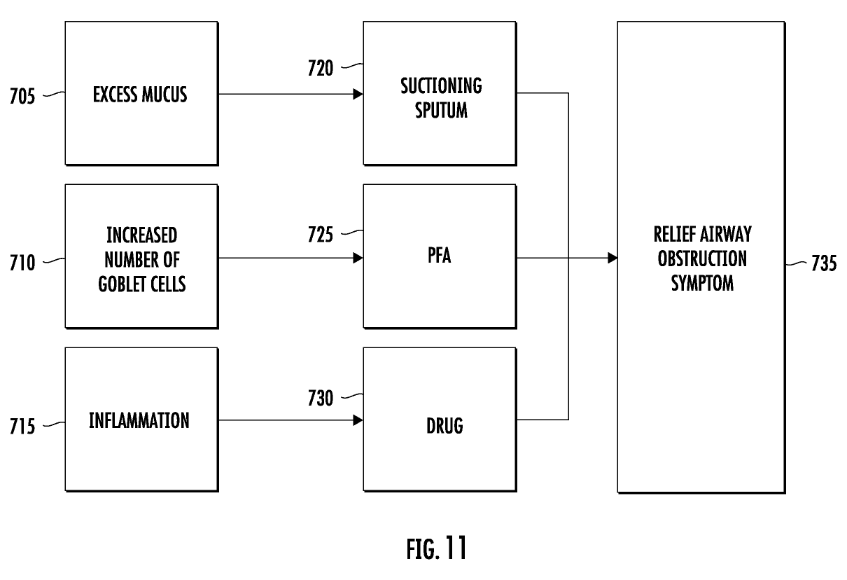

FIG. 11 presents a flow chart of exemplary methods applicable to any of the examples and embodiments of the present disclosure. At block 705, excess mucus may be seen and/or discovered through an imaging technique such as endoscopy, fluoroscopy, CT, ultrasound and/or the like. These procedures and methods may also reveal an increased number of goblet cells (shown in block 710) which is a key indicator of inflammation as depicted in block 715. In this and other examples, methods may include suctioning sputum (as depicted in block 720) through a suctioning means and this may be achieved within a patient or subject’s respiratory system.

Suctioning means may include, but are not limited to aspirators, vacuums, pumps, known suctioning devices, and/or any combination or permutation of the aforementioned. Prior to, following and/or concurrent with the suctioning step as depicted in block 720, pulsed-field-ablation (i.e., any of the ablation therapies described herein) may be applied to the patient and/or subject treatment site as depicted in block 725. Prior to, concurrently and/or subsequently, a drug, therapeutic, agent or the like may be delivered to the treatment site as depicted in block 730, resulting in relief of an airway obstruction, and/or airway obstruction symptom as depicted in block 735.

In other non-limiting examples, methods may include methods of treating respiratory maladies and/or malformations, which may include: deploying a first catheter into a lung of a patient or subject; suctioning sputum from a lung of a patient or subject by a suctioning means (e.g., vacuum, pump, aspirator, etc.); activating one or more electrodes disposed on or within the first catheter, thereby delivering pulsed electrical therapy for ablation of a treatment site within a lung (or related structure) of a patient or subject; and delivering a drug and/or therapeutic to the treatment site.

Alternatively, or additionally, methods of the present disclosure may include delivering a drug and/or therapeutic and/or agent and/or anti-inflammatory to the treatment site by delivering one or more drugs and/or therapeutics and/or agents and/or anti-inflammatories via one or more lumens within the first catheter and/or subsequent catheters and/or like devices.

Alternatively, or additionally, methods of the present disclosure may include delivering one or more drugs and/or therapeutics to the treatment site via the first catheter (or like device); whereby the one or more drugs and/or therapeutics are selected from the group comprising: one or more drug and/or therapeutic coatings, one or more drug and/or therapeutic dispensing elements, one or more lumen-delivered drugs and/or therapeutics (i.e., drugs and/or therapeutics delivered through an ablation catheter or catheter through one or more lumens), one or more electrically delivered drugs and/or therapeutics, and one or more pulse-delivered drugs and/or therapeutics.

In this and other examples, the delivery of one or more anti-inflammatories, drugs, therapeutics and/or agents may be performed after, prior to, or concurrent with ablation.

In yet other non-limiting examples, in the methods and systems disclosed herein, two or more electrodes may be disposed upon and/or within an expandable scaffolding that may be operably and/or communicably coupled to both the first catheter and a control knob coupled to the first catheter. It is also contemplated that methods may include spreading apart the two or more electrodes (i.e., increasing the relative distance between two or more electrodes) via an expandable scaffolding that is operably coupled to the first catheter and in response to action and/or activation and/or actuation of a control knob operably coupled to the first catheter.

This disclosure is, in many respects, only illustrative. Changes may be made in details and arrangement of steps without exceeding the scope of the disclosure. This may include, to the extent that it is appropriate, the use of any of the features of one example embodiment being used in other embodiments.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed is:

1. An ablation catheter, comprising:
a proximal shaft including an expandable scaffolding;
a control knob;
a distal shaft including a distal tip disposed at a distal end of the distal shaft;
wherein the control knob is disposed proximate the proximal shaft and the distal shaft;
wherein the ablation catheter includes one or more electrodes; and
wherein both the proximal shaft and the distal shaft include at least one lumen configured to receive the one or more electrodes therein.

2. The ablation catheter of claim 1, wherein the one or more electrodes comprise three electrodes and wherein the proximal shaft and the distal shaft each include three lumens configured to receive the three electrodes therein.

3. The ablation catheter of claim 1, wherein the proximal shaft and the distal shaft each include one or more lumens configured to pass one or more drugs or one or more therapeutics therethrough.

4. The ablation catheter of claim 1, wherein the distal tip includes one or more electrodes disposed proximate thereto and/or thereon.

5. The ablation catheter of claim 1, wherein the expandable scaffolding includes an expandable balloon.

6. The ablation catheter of claim 1, wherein the expandable scaffolding includes one or more electrodes disposed within and/or upon the expandable scaffolding.

7. The ablation catheter of claim 1, wherein the expandable scaffolding includes one or more drug coatings disposed thereon and/or one or more drug dispensing elements disposed thereon or within.

8. The ablation catheter of claim 1, wherein the ablation catheter is configured to be operably connected to an electrical pulse generator, such that the ablation catheter is configured to deliver pulsed electrical energy through the one or more electrodes.

9. The ablation catheter of claim 1, wherein the one or more electrodes are disposed in an array of electrodes and wherein the array of electrodes is one or more of a spiral array, a linear array, a curvilinear array, and an interrupted array.

10. The ablation catheter of claim 5, wherein the expandable balloon includes one or more of one or more electrodes and one or more drug dispensing members.

11. The ablation catheter of claim 6, wherein the expandable scaffolding includes one or more electrodes disposed upon and/or within the expandable scaffolding in an array selected from one or more of: a spiral array, a linear array, a curvilinear array and an interrupted array.

12. A method of treating respiratory maladies and malformations, comprising:
deploying a first catheter into a lung of a patient or subject;
suctioning sputum from a lung of a patient or subject by a suctioning means;
activating one or more electrodes disposed on or within the first catheter, thereby delivering pulsed electrical therapy for ablation of a treatment site within a lung of a patient or subject; and
delivering a drug and/or therapeutic to the treatment site.

13. The method of claim 12, wherein delivering a drug and/or therapeutic to the treatment site comprises delivering one or more drugs and/or therapeutics via one or more lumens within the first catheter.

14. The method of claim 27, further comprising delivering one or more drugs and/or therapeutics to the treatment site via the first catheter; wherein the one or more drugs and/or therapeutics comprise: one or more drug and/or therapeutic coatings, one or more drug and/or therapeutic dispensing elements, one or more lumen-delivered drugs and/or therapeutics, one or more electrically delivered drugs and/or therapeutics, or one or more pulse-delivered drugs and/or therapeutics.

15. The method of claim 12, further comprising delivering anti-inflammatory drugs and/or therapeutics subsequent to or concurrent with ablation.

16. The method of claim 12, wherein the one or more electrodes comprise two or more electrodes disposed upon and/or within an expandable scaffolding operably coupled to both the first catheter and a control knob operably coupled to the first catheter;
the method further comprising spreading apart the two or more electrodes via an expandable scaffolding operably coupled to the first catheter and in response to action of a control knob operably coupled to the first catheter.

17. The method of claim 13, further comprising: delivering one or more drugs and/or therapeutics to the treatment site concurrent with or subsequent to delivering pulsed electrical therapy for ablation of a treatment site within a lung of a patient or subject.

18. A pulsed-electric-field ablation catheter, comprising:
a proximal shaft;
a control knob;
a distal shaft including a distal tip disposed at a distal end of the distal shaft;
wherein the control knob is disposed proximate the proximal shaft and the distal shaft;
wherein the ablation catheter includes one or more electrodes disposed upon or within an expandable framework;
wherein the expandable framework further includes one or more drug coatings or one or more drug dispensing elements disposed upon or within the expandable framework; and
wherein both the proximal shaft and the distal shaft include at least one common lumen configured to receive the one or more electrodes, one or more drugs, and/or one or more therapeutics therethrough.

19. The pulsed-electric-field ablation catheter of claim 18, wherein the control knob includes one or more locking members.

20. The pulsed-electric-field ablation catheter of claim 18, wherein the expandable framework includes one or more electrodes disposed upon and/or within the expandable framework in an array selected from one or more of: a spiral array, a linear array, a curvilinear array and an interrupted array.
